(19) United States
(12) Patent Application Publication
(10) Pub. No.: US 2005/0014208 A1
(43) Pub. Date: Jan. 20, 2005

(54) Method and kit for diagnosing or controlling the treatment of breast cancer

(76) Inventors: Alf-Andreas Krehan, Anzing (DE); Pia Steffens, Hannover (DE); Bertha Gutierrez, Hannover (DE); Stefanie Waschuetza, Hannover (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/488,828

(22) PCT Filed: Sep. 6, 2002
(86) PCT No.: PCT/EP02/09999

(30) Foreign Application Priority Data
Sep. 6, 2001 (DE) 101 43 776.5
May 17, 2002 (EP) PCT EP02 05489

(51) Int. Cl. G01N 33/574; C12M 1/34

(52) U.S. Cl. 435/7.23; 435/287.2

(57) ABSTRACT

The present invention pertains to a procedure and a kit for the diagnosis or monitoring of breast cancer in humans. This procedure is based on the feature that one recognizes the presence or absence in a human blood sample of at least two different mRNAs that code for various members of the tumor marker proteins EGF-R, CEA, stanniocalcin, CK20, MAGE-3, GA733.2, MUC1, Her-2/neu, claudin-7, and/or PDGF-β, and a conclusion is drawn from this in regard to the presence of mammary carcinoma cells in the blood sample, and thus in regard to possible metastasis.

The present invention pertains to a procedure and a kit for the diagnosis or monitoring of breast cancer in humans.

The ability to detect a relapsing malignant tumor in a timely manner via the occurrence of metastasizing tumor cells in the blood is relevant in cancer after-care. So-called “tumor markers” are determined quantitatively at the protein level (immunologically or enzymatically) in the blood, or in other bodily fluids, in cancer patients when using current test methods.

These detection procedures are suitable only to a limited extent for tumor diagnosis, or monitoring/after-care, since increased tumor marker values can also be produced by nontumor diseases (e.g. inflammation of the gastrointestinal tract, cirrhosis of the liver, viral infections), heavy smoking, or as a result of pregnancy.

Breast cancer is the most frequent diagnosis when a tumor disease is found in women (26.4% of all new diseases). Despite massive efforts that are being made in regard to early detection, treatment and after-care, this disease still ranks first in cancer-related deaths in women. The number of cases of this disease in the western industrialized countries has been increasing further over past years despite the intensified efforts in regard to early detection. The high rate of metastasis following the initial treatment is problematical, leading to the death of the patient after only 1-3 years in the majority of cases. The main reason for this is the spread of tumor cells in the early stages of tumor development. Thus, in addition to the initial recognition of a mammary carcinoma, the earliest possible detection of metastasizing cells is of especially decisive importance for a successful treatment. Likewise, a definitive negative detection can be helpful in clinical stage I when one must decide whether the patient is to be subjected to the stress of chemotherapy or an operation.

The currently used diagnostic methods are inexact when one is dealing with the evaluation of the malignant potency of residual tumors after chemotherapy has been carried out in the metastasizing stages. Some clinical studies indicate the prognostic importance of disseminated tumor cells. However, numerous methodological aspects are critical and have not been adequately standardized so far. Thus, detection methods for occult-or residual-metastasis have to be found that permit timely classification into the various primarily curative therapeutic options.

The effort to improve the chances of healing is currently accompanied, on the one hand, by the search for and the use of new tumor markers and, on the other hand, by increases in sensitivity in the methods that are used.

The problem of the present invention is to make available a procedure and a kit with which the diagnosis or monitoring of breast cancer is possible in a simple, safe and repeatable manner.

This problem is solved by the procedure in accordance with claim 1 and with the kit in accordance with claim 30 and the microarray in accordance with claim 50. Advantageous further developments of the procedure, of the kit, and of the microarray are given in the pertinent dependent claims.

In accordance with the invention, the presence or absence of mRNA from the tumor marker proteins EGF-R, CEA, CK20, MAGE-3, GA733.2, MUC-1≅GA15.3, Her-2/neu, claudin-7, PDGF-β and/or stanniocalcin are recognized in a human blood sample by means of the kit in accordance with the invention or by means of the procedure in accordance with the invention.

Since the RNAs of the markers that have been described are not normally present in expressed form in the blood of healthy persons, a direct correlation is found between a positive RT-PCR result detection for these tumor markers and circulating tumor cells in the blood that can lead to metastasis.

Since individual markers can be expressed differently in a therapy-dependent manner, and breast cancer exhibits pronounced heterogeneity in the expression pattern of the breast cancer cells, it is expedient to examine a combination of tumor markers in order to recognize all the tumor cells that are circulating in the blood. As a result of this, tumor cells can also be recognized when the expression of a particular marker is relatively slight in a patient or in a stage of the disease, which could otherwise lead to a supposedly negative result. However, the use of additional markers usually encounters limits if mononuclear blood cells exhibit background expression (“illegitimate transcription”) that impedes exact analysis.

Thus the following combination of markers is proposed in accordance with the invention for the recognition of breast cancer cells.

Two of the markers from the following groups
GA733.2 and MUC1;
Her-2/neu and claudin-7;
CK20, MAGE-3 and MUC 1; or
stanniocalcin, EGFR and CEA or
the following combinations
GA733.2, MUC1, Her-2/neu, claudin-7;
GA733.2, PDGF-β, Her-2/neu, claudin-7;
GA733.2, MUC1, CEA;
GA733.2, PDGF-β, claudin-7; or
GA733.2, MUC1 and claudin-7.

Use can be made of the primers indicated in the following table for the amplification of segments of the markers.

Markers for Mammary Carcinoma

| | | |
|---|---|---|
| Primername | Sequence 5′ → 3′ | PCR-Product |

Tumor marker

| | | |
|---|---|---|
| GA733.2 sense | AATCGTCAATGCCAGTGTACTTCA | 395 bp |
| GA733.2 antisense | TAACGCGTTGTGATCTCCTTCTGA | |
| EGFR sense | AGTCGGGCTCTGGAGGAAAAGAAA | 163 bp |
| EGFR antisense | GATCATAATTCCTCTGCACATAGG | |
| CEA sense | AGAAATGACGCAAGAGCCTATGTA | 231 bp |
| CEA antisense | AACTTGTGTGTGTTGCTGCGGTAT | |
| MUC1 sense | TCAGCTTCTACTCTGGTGCACAAC | 299 bp |
| MUC1 antisense | TGGTAGTAGTCGGTGCTGGGATCT | |
| Her-2 sense | CCCAGTGTGTCAACTGCAGCCAGT | 265 bp |
| Her-2 antisense | CAGATGGGCATGTAGGAGAGGTCA | |
| Claudin-7 sense | GTCTTGCCGCCTTGGTAGCTTGCT | 225 bp |
| Claudin-7 antisense | TGGACTTAGGGTAAGAGCGGGGTG | |
| CK20 sense | ATCTCCAAGGCCTGAATAAGGTCT | 336 |
| CK20 antisense | CCTCAGTTCCTTTTAATTCTTCAGT | |
| MAGE3 sense | CTCCAGCCTCCCCACTACCATGAA | 375 bp |
| MAGE3 antisense | TTGTCACCCAGCAGGCCATCGTAG | |
| Stanniocalcin sense | AACCCATGAGGCGGAGCAGAATGA | 254 bp |
| Stanniocalcin antisense | CGTTGGCGATGCATTTTAAGCTCT | |
| PDGF-β sense | TCTCTCTGCTGCTACCTGCGTCTG | |
| PDGF-β antisense | GTTGGCGTTGGTGCGGTCTATGAG | |

Internal Control

| | | |
|---|---|---|
| Actin Sense | CTGGAGAAGAGCTACGAGCTGCCT | 111 bp |
| Actin antisense | ACAGGACTCCATGCCCAGGAAGGA | |

The designations of the markers are explained in the following section:

| | | Alternative |
|---|---|---|
| Gene or gene product | Gene | designation |
| Human carcinoma-associated antigen | GA733-2 | GA733.2 |
| GA733-2 gene | | |
| Human epidermal growth factor recep- | EGFR | EGFR |
| tor (EGFR) gene | | |
| Human carcinoembryonic antigen | CEA | CEA |
| (CEA) gene | | |
| Homo sapiens mucin 1 (MUC1) | MUC1 | CA15-3 |
| Homo sapiens C-erb B2/neu protein | HER-2/neu | HER-2 |
| (ERBB2) gene | | |
| Homo sapiens claudin 7 (CLDN7), | claudin7 | Claudin-7 |
| mRNA | (CLDN7) | |
| Homo sapiens gene for cytokeratin 20 | CK20 | CK20 |
| Human MAGE-3 antigen (MAGE-3) | MAGE-3 | MAGE-3 |
| gene | | |
| Homo sapiens stanniocalcin 1 (STC1) | Stanniocalcin | Stanniocalcin |
| gene | (STC1) | |
| Platelet derived growth factor-β | PDGF-β | PDGF-β |

In the case of using RT-PCR systems for the detection of tumor cells, specificity is a critical point because of the very high amplification rate. The least contamination, e.g. via extraneous RNA or illegitimate transcription, can in this way falsify the result.

An increase in specificity as a result of concentrating the tumor cells relative to the blood cells and, at the same time, an increase in sensitivity in the detection of tumor cells can be achieved (detection rate of 1 tumor cell per 10⁷ mononuclear blood cells) by using immunocytochemistry with monoclonal antibodies that act against tumor cell antigens. In this way the tumor cells are separated by means of specific antibodies or an antibody mixture of mononuclear blood cells. Separation can take place by means of magnetic particles (Dynal) to which the antibodies are bound. This is described in greater detail in the following segment.

Eukaryotic cells carry a plurality of different molecules on their cell surface. The combination of expressed surface molecules differs depending on the origin and the function of the individual cell, so that patterns are produced that are cell-type specific. Antibodies are utilized in order to recognize these cell-type specific patterns. Antibodies bind with high specificity to their antigen, namely to selected surface molecules in this case. This property is utilized in order to recognize cells and to differentiate them from one another by means of specific antibody binding on the basis of their cell-type specific patterns.

The expression of special surface proteins differentiates tumor cells from nontransformed cells of this cell type. Since, in the case of tumor cells, this special pattern of surface antigens also differs from the patterns that are typical of blood cells, tumor cells can be differentiated in the blood. In order to identify tumor cells, antibodies that specifically recognize these special surface proteins are utilized as tools. This specific antibody binding becomes usable for various analyses and separation methods.

In addition to recognizing cells via their surface epitopes, it is also possible to separate recognized cells from nonrecognized ones because of the intensive binding of immunoglobulins that are specially selected for this purpose.

1. Separation Principle Based on the Liquid Phase; e.g. Continuous Flow Cytometry:

Antibodies are coupled to fluorescent dyes for the purpose of continuous flow cytometry analysis. Isolated cells are individually led past a light source (laser) in a constant stream of liquid. The fluorescent dyes bound to the antibodies are excited during illumination of the cells, and they irradiate light of a particular wavelength. The irradiated light is detected, and the measured signal is stored in digital form. The light signal can be assigned to individual cells. The antibody-labeled cell is recognized in this way, and can now be separated from other cells. The cells are isolated in extremely small drops for separation purposes. After recognizing the antibody-labeled cell, the drops in question are deflected into a collection container.

2. Separation Principle Based on the Solid Phase; e.g. Magnetic Separation:

Antibodies are coupled to pseudomagnetic particles for the purpose of magnetic separation. After introducing the pseudomagnetic particles to a magnetic field, the particles migrate in this magnetic field. During movement in this magnetic field, the cells to which the coupled antibodies are bound are carried along, and separated from other cells.

Thus, in order to recognize tumor cells by means of magnetic particles, antibodies are covalently coupled to pseudomagnetic particles that possess a defined number of chemically activated sites on their surface. The separation specificity is determined by the specificity of the antibodies. A blood sample that contains tumor cells is mixed with antibody-coupled magnetic particles; the particles and blood then move relative to one another. Those (tumor) cells which are recognized by the antibodies (that are bound to the solid phase) and which are firmly bound to them follow the movement of the particles. As a result, it is possible to withdraw, from the blood, the particles with the cells that are bound to them (e.g. toward the wall of the separation vessel) upon applying a magnetic field. The blood that has been tumor-cell depleted in this way can be exchanged for other solutions, whereby the cells that have been separated via magnetic particles remain behind, and are available for additional applications until the point in time of switching off/removing the magnetic field.

Use can advantageously be made of specific antibody mixtures in order to recognize the tumor cells, whereby these mixtures have either been optimized with respect to tumor cells in a general manner, or they have been specifically optimized with respect to breast cancer cells as well. For example, a combination of MOC-31 antibodies (Novocastra) and Ber-EP-4 antibodies (DAKO) is suitable for recognizing tumor cells in the blood.

Recognition that is specially directed toward breast cancer cells can be achieved via an additionally optimized antibody mixture in accordance with the table below. This is based on the selective expression of certain surface proteins, whereby breast cancer cells are differentiated from other cancer cells.

| Antigen | Clone | Concentration |
|---|---|---|
| Epith. Membr. Antigen | 131-1174 (Hiss) | 0.625 μg/10⁶ cells |
| Epith. Membr. Antigen | E29 (DAKO) | 0.25 μg/10⁶ cells |
| Epith. Membr. Antigen | GP1.4 (Novocastra) | 18.75 μg/10⁶ cells |
| MUC-1 | HMPV.2 (Pharmingen) | 1.25 μg/10⁶ cells |

In comparison to the antibodies when used separately in each case, such antibody mixtures show increased sensitivity in terms of cell recognition and cell separation, independent of the method used.

Some examples, in accordance with the invention, of detection procedures for breast cancer cells in blood samples will be described in the following segment.

The following aspects are shown:

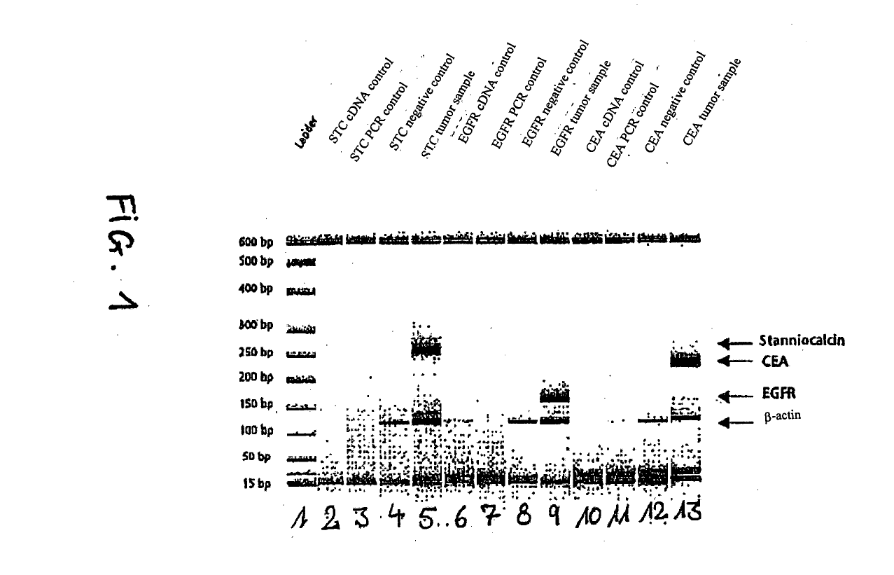

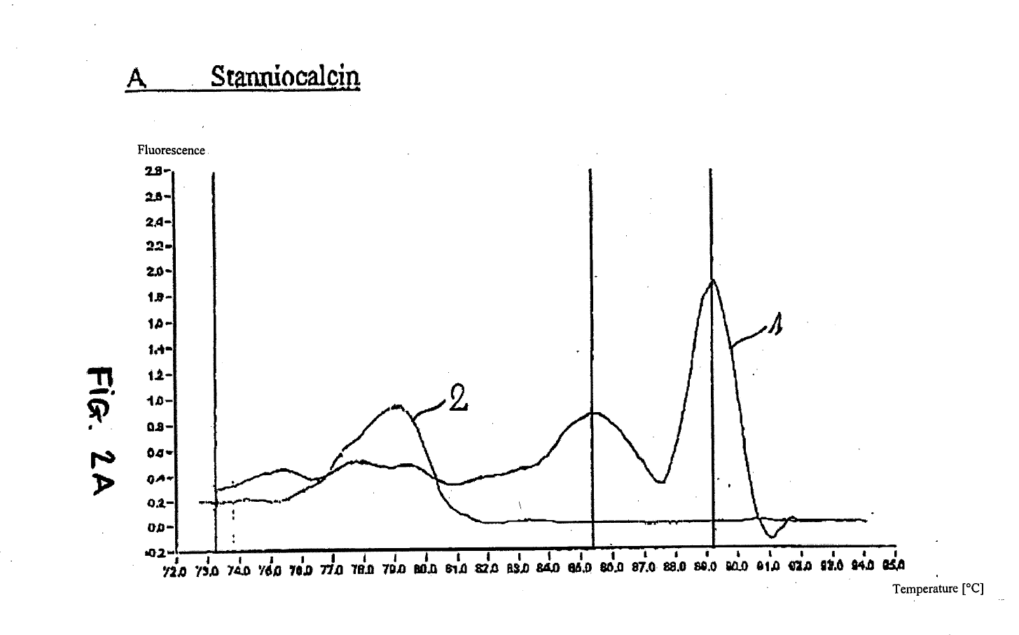

FIG. 1 shows the detection of PCR products via electrophoresis;

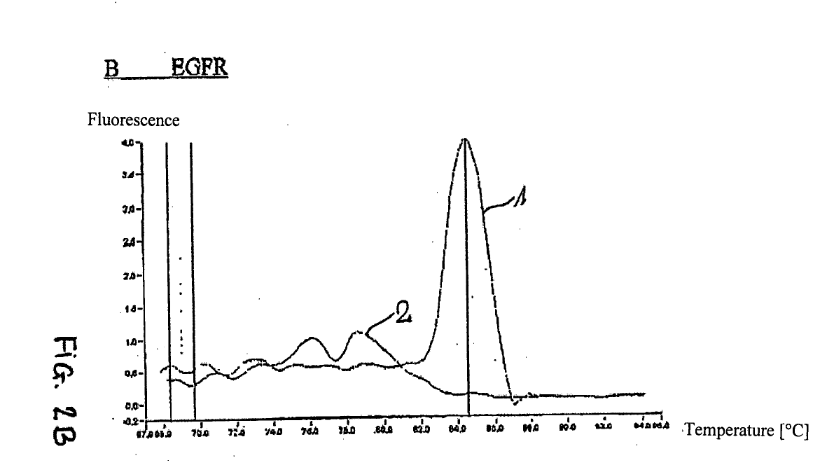

FIGS. 2A-C show tumor marker detection by means of a Light Cycler;

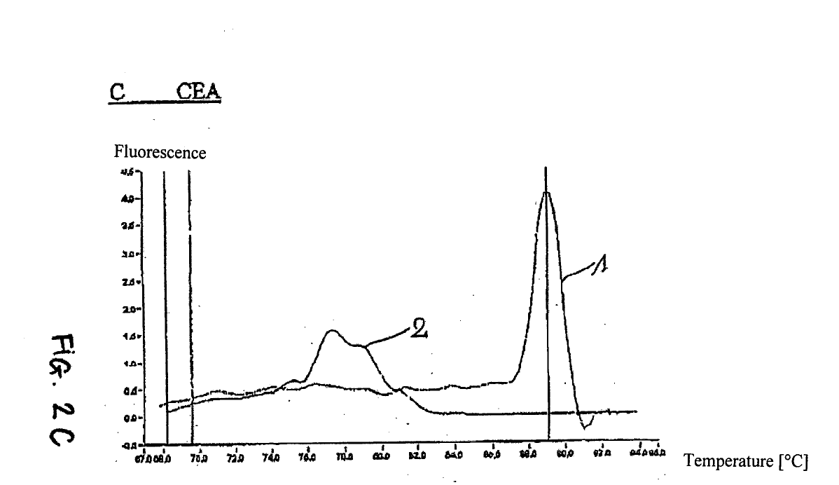

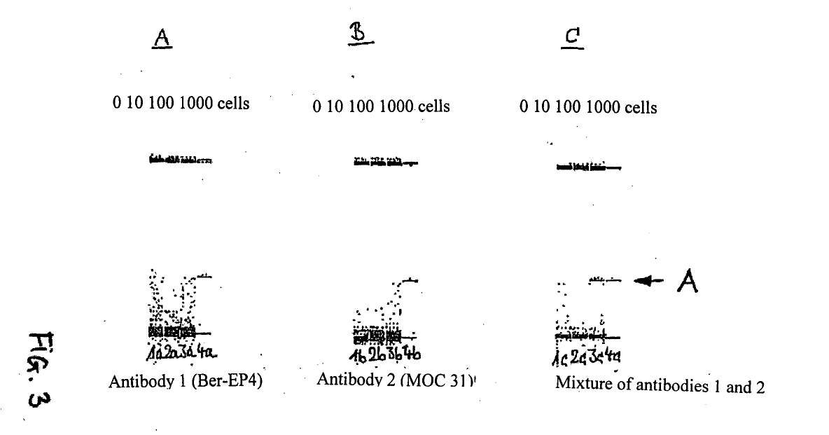

FIG. 3 shows the detection of cell separation by means of antibody-labeled magnetic particles;

FIGS. 4-8 show additional examples of the detection of mammary carcinoma cells by means of several tumor markers.

In the first example, the RNA from 1 mL of EDTA/whole blood was processed using the QIAamp RNA Blood Mini Kit (Qiagen, Hilden). Contamination by genomic DNA was avoided via additional DNA digestion in the column using an RNA-free DNase Set (Qiagen, Hilden).

The processing of the RNA from 1 mL of EDTA/whole blood was verified photometrically via the 260:280 nm ratios. For the purposes of quality and quantity determinations in this connection, 1 μL of the mixture can be analyzed via electrophoretic separation on an RNA 6000 chip using the Agilent Bioanalyzer 2100.

The isolated RNA was denatured in an appropriate volume together with oligo(dT) 15 primers (Promega, Mannheim) for 5 min at 65° C., and then incubated directly on ice. cDNA synthesis took place by means of the Sensiscript™ Reverse Transcriptase Kit (Qiagen, Hilden) in 20 μL of reaction mixture in accordance with Table 1 for 1 h at 37° C. with subsequent reverse transcriptase inactivation for 5 min at 95° C. that was then followed by cooling on ice.

TASBLE 1
cDNA synthesis components

| Components | Volume | Final concentration |
|---|---|---|
| RNA | x μl | 5 ng/μl |
| 10 x RT buffer | 2 μl | 1x |
| dNTP mixture (5 mM in each case) | 2 μl | 0.5 mM in each case |
| Oligo (dT)-Primer (10 μM) | 2 μl | 1 μM |
| Rnase-Inhibitor | 1 μl | 0.5 Units/μl |
| Reverse transciptase | 1 μl | 4 U |
| RNase-free water | ad 20 μl | |

Using the cDNA that was produced in this way, a multiplex PCR was carried out for each of the selected tumor markers stanniocalcin, EGF-R, and CEA and also for β-actin as an internal control. The PCR mixture is illustrated in Table 2 that follows.

TABLE 2
PCR mixture

| Components | | Volume | Final concentration |
|---|---|---|---|
| | CDNA | 6 μl | |
| | 10 x PCR buffer* | 5 μl | 1x |
| | dNTP mixture | 1 μl | 200 μM in each case |
| | Primer | (See Table 3) | |
| | DMO addition*** | 1.0 μl | |
| | Taq-DNA | 0.5 μl | 2.5 U |
| | Polymerase** | | |
| | H₂O | ad 50 μl | |

(*contains 15 mM MgCl₂;
**HotStarTaq ™ DNA polymerase; Qiagen, Hilden
***DMSO azddition in the case of stanniocalcin)

A primer pair, which is seen in Table 3 below, was used for each tumor marker in this regard.

TABLE 3
List of PCR primers

| | | |
|---|---|---|
| | | PCR- |
| Primername | 5′ → 3′ sequence | product |

Tumor marker

| | | |
|---|---|---|
| Stanniocalcin | AACCCATGAGGCGGAGCAGAATGA | 254 bp |
| sense | | |
| Stanniocalcin | CGTTGGCGATGCATTTTAAGCTCT | |
| antisense | | |
| EGF-R sense | AGTCGGGCTCTGGAGGAAAAGAAA | 163 bp |
| EGF-R antisense | GATCATAATTCCTCTGCACATAGG | |
| CEA sense | AGAAATGACGCAAGAGCCTATGTA | 231 bp |
| CEA antisense | AACTTGTGTGTGTTGCTGCGGTAT | |

Internal control

| | |
|---|---|
| β-actin sense | CTGGAGAAGAGCTACGAGCTGCCT |
| β-actin antisense | ACAGGACTCCATGCCCAGGAAGGA |

The primer combinations and quantities that were used for the individual tumor marker detections are listed in Table 4 that follows.

TABLE 4

List of primer quantities and
primer combinations

| Marker | | | |
|---|---|---|---|
| Primer | Stanniocalcin | EGF-R | CEA |
| Stanniocalcin | 25 pmol | | |
| sense | | | |
| Stanniocalcin | 25 pmol | | |
| antisense | | | |
| EGF-R sense | | 25 pmol | |
| EGF-R antisense | | 25 pmol | |
| CEA sense | | | 25 pmol |
| CEA antisense | | | 25 pmol |
| β-actin sense | 1 pmol | 1 pmol | 1 pmol |
| β-actin antisense | 1 pmol | 1 pmol | 1 pmol |

The PCR was carried out using the conditions indicated in Table 5 together with the marker-specific melting temperatures and numbers of cycles indicated in Table 6.

TABLE 5
PCR conditions

| | | | | |
|---|---|---|---|---|
| Vorabdenaturierung | | 95° C. | 15 min | |
| ZYklus | | | | |
| | 1. Denaturierung | 94° C. | 1 min | |
| | 2. Annealing | x° C. | 1 min | (s. Table 6) |
| | 3. Extension | 72° C. | 1 min | |
| Finale | | 72° C. | 10 min | |
| Extension | | | | |
| | | 4° C. | Pause | |

TABLE 6
Marker-specific annealing temperature and number of cycles

| Marker | | | |
|---|---|---|---|
| Annealing | Stanniocalcin | EGF-R | CEA |
| Temperature | 58° C. | 64° C. | 60° C. |
| Number of cycles | 35 | 35 | 40 |

1 μL of the PCR product produced in this way was separated in an Agilent Bioanalyzer 2100 on a DNA chip (500), and the result of the separation was documented electronically. The results are shown in FIG. 1. In this diagram, lane 1 shows a 100 kb ladder, and lanes 2-13 show the results for, the corresponding samples. As can be seen, lane 5 shows a PCR product for the tumor marker stanniocalcin; lane 9 shows a PCR product for the tumor marker EGF-R, and lane 13 shows a PCR product for the tumor marker CEA, whereas all samples with a biological material in accordance with lanes 4, 5, 8, 9, 12 and 13 contain PCR products for the internal control β-actin.

Lanes 2, 3, 6, 7, 10, 11 do not contain any biological material, so that no corresponding PCR products arise there. In FIG. 1, the so-called cDNA control is a mixture totally without RNA; the so-called PCR control is a mixture without cDNA, and the negative control is a mixture containing RNA from a healthy control person. In FIG. 1, CEA stands for carcinoembryonic antigen; STC stands for stanniocalcin, and EGF-R stands for epidermal growth factor receptor.

FIG. 2 shows an alternative analysis by means of fluorescence-based real time PCR using intercalating fluorescent dyes.

As an alternative to block PCR, this tumor marker detection procedure can also be done by means of a Light Cycler (Roche, Basel).

Reverse transcription of the mRNA was done as described above. The PCR was then carried out with the Light Cycler DNA Master Sybr Green I′ Kit (Roche, Basel) in accordance with data from the manufacturer under conditions that had been optimized for each tumor marker. The oligonucleotides that are indicated in Table 3 were used as primers in this case. Table 7 and Table 8, respectively, show the mixture for the PCR and the PCR conditions using the Light Cycler.

TABLE 7
PCR mixture: Light Cycler

| Tumor marker | | | |
|---|---|---|---|
| Components | Stanniocalcin | EGF-R | CEA |
| CDNA | 3.0 μl | 3.0 μl | 3.0 μl |
| MgCl₂ | 3.0 mM | 3.5 mM | 3.5 mM |
| Primer | 0.5 μM | 0.5 μM | 0.5 μM |
| Light Cycler- | 2 μl | 2 μl | 2 μl |
| DNA Master | | | |
| Sybr Green | | | |
| DMSO | 1 μl | — | — |
| H₂O | ad 20 μl | azd 20 μl | ad 20 μl |

TABLE 8
PCR conditions: Light Cycler

| | Stanniocalcin | EGFR | CEA |
|---|---|---|---|
| Denaturation | 95° C., 30 Sec | 95° C., | 95° C., |
| | 20° C./Sec | 30 Sec | 30 Sec 20° C./Sec |
| | | 20° C./Sec | |
| Amplification | 95° C., 5 Sec, | 95° C., | 95° C., |
| | 20° C./Sec | 5 Sec, | 5 Sec, |
| | | 20° C./Sec | 20° C./Sec |
| | 67° C., 10 Sec, | 60° C., | 60° C., |
| | 20° C./Sec | 10 Sec, 20° C./ | 10 Sec, |
| | | | 20° C./Sec |
| | 73° C., 15 Sec, | 73° C., | 72° C., |
| | 5° C./Sec | 15 Sec, | 12 Sec, |
| | | 5° C./Sec | 5° C./Sec |
| Melting Curve | 95° C., 0 Sec, | 95° C., | 95° C., |
| | 20° C./Sec | 0 Sec, | 0 Sec, |
| | | 20° C./Sec | 20° C./Sec |
| | 70° C., 20 Sec, | 65° C., | 65° C., |
| | 20° C./Sec | 20 Sec, | 15 Sec, |
| | | 20° C./Sec | 20° C./Sec |
| | 95° C., 0 Sec, | 95° C., | 95° C., |
| | 0, 1° C./Sec | 0 Sec, | 0 Sec, |
| | | 0, 1° C./Sec | 0, 1° C./Sec |
| Cooling | 30° C., 30 Sec | 30° C., | 30° C., |
| | 20° C./Sec | 30 Sec | 30 Sec |
| | | 20° C./Sec | 20° C./Sec |

The result of this PCR and the evaluation using Light Cycler technology are illustrated in FIGS. 2A through 2C. The control curve is designated 2 in all the FIGS. 2A through 2C, whereas the curve that was recorded for the sample is designated 1.

In this analysis, the melting curve for the PCR products is analyzed by means of the Sybr Green I detection method. The pertinent graph in FIGS. 2A through 2C is the fluorescence that was measured as a function of the temperature. The fluorescence peaks that occur in the control mixtures are attributable to primer dimers.

FIG. 2A represents the melting curve analysis of the stanniocalcin PCR product. The melting point of the main product is 89.2° C., and the melting point of the secondary product is 85.3° C. Such fluorescence peaks cannot be seen in the case of the control sample.

FIG. 2B shows the melting curve analysis of the EGFR PCR product with a melting point of 84.6° C.

FIG. 2C shows the melting curve analysis of the CEA PCR product with a melting point of 89.06° C.

As an alternative to the methods that are illustrated here, use can, of course, be made of conventional methods of analysis as well, such as agarose gel electrophoresis in which, for example, 25 μL of the PCR product synthesized above are separated over a 2.5% agarose gel, and the DNA bands are then stained with ethidium bromide and rendered visible. Documentation can be carried out with the help of e.g. the DUO Store System from Intas.

In addition, fragment analysis by means of the ABI Prism 310 Genetic Analyzer (Applied Biosystems, Weiterstadt) can also be used for the evaluation. In order to do this, a PCR is carried out with fluorescence-labeled primers and then, for example, 1 μL in each case of each PCR product is used at a dilution of 1:50.

Detection by means of sequence-specific fluorescence-labeled hybridization samples is possible as an additional detection procedure, whereby these samples allow the evolution of products to be monitored after each PCR cycle. A conclusion can then be drawn on the basis of special standards in regard to the quantity of starting RNA.

The enrichment of the cell fraction which is used for this purpose and which arises from the blood sample used is central for the quality of the RNA, which is isolated as the basis of the detection procedure, and the cDNA that is synthesized therefrom. Four different methods are available for this as follows.

a) Enrichment by Means of Repeated Centrifugation Following Erythrocyte Analysis:

1 mL of EDTA/blood is lysed for 20 min on ice following the addition of 5 volumes of erythrocyte lysis buffer (“QIAmp Blood Kit,” Qiagen; Hilden). The plasmallysate is removed from the pelletized cells and resuspended, and then renewed centrifugation takes place for 20 min at 3000×g. After removing the supernatant liquor, the pelletized leukocyte fraction is available for RNA preparation.

b) Enrichment by Means of Density Gradient Centrifugation:

Cells of different mean volume-based density can be separated from one another via a density gradient that is produced by means of centrifugation. Mononuclear blood cells are separated by means of a Ficoll-Hypaque gradient (Pharmacia, Uppsala, Sweden), and then washed twice with PBS/1% FCS.

c) Enrichment of Tumor Cells by Means of FACS Continuous Flow Cytometry:

The mononuclear cells from the fraction enriched under b) are incubated with fluorescence-labeled mononuclear antibodies that act against tumor-specific surface proteins. The labeled cells are washed twice with PBS, and then 10⁷ cells are resuspended in 1 mL of PBS. A FACS Vantage SE continuous flow cytometer (Becton Dickinson) is used in order to isolate the tumor cells. Data recording, instrument control, and data evaluation are done via the CellQuest program. The sorted cells are transferred to a 1.5-mL reaction vessel (filled with 1 mL of PBS). The RNA can then be isolated as described above.

As an alternative, the isolated fraction of mononuclear blood cells, which were isolated in accordance with one of the above procedures, was lysed in trizole reagent (Gibco BRL, New York, USA), and homogenized by means of a pipette. Following chloroform extraction, the RNA-containing aqueous phase is precipitated in isopropanol at −80° C. After washing twice in 80% ethanol, the pellet is dried in air, and then resuspended in RNase-free water.

Reverse transcription and mRNA detection as described above then follow on from this isolation of the RNA.

d) Enrichment of Tumor Cells by Means of Immunomagnetic Separation:

The expression of special surface proteins differentiates tumor cells from nontransformed cells of this cell type. Since, in the case of tumor cells, this special pattern of the surface antigens is also different from the patterns that are typical of blood cells, one can differentiate between tumor cells in the blood. In order to identify tumor cells, antibodies which specifically recognize these special surface proteins are utilized as tools. Specific antibody binding is made usable for the procedure in accordance with the invention. Antibodies are covalently coupled to pseudomagnetic particles that possess a defined number of chemically activated sites on their surface. The separation specificity is determined by the specificity of the antibodies. A blood sample that contains tumor cells is mixed with antibody-coupled magnetic particles; two different mixtures of antibodies are used as antibodies in the various examples; the particles and blood then move relative to one another, e.g. by means of “over-end rotators” in samples that are located in a closed container or by means of alternating magnetic fields. Those (tumor) cells which are recognized by the antibodies (that are bound to the solid phase) and which are firmly bound to them follow the movement of the particles. As a result, it is possible to withdraw, from the blood, the particles with the cells that are bound to them (e.g. toward the wall of the separation vessel) upon applying a magnetic field. The blood that has been tumor cell depleted in this way can be exchanged for other solutions, whereby the cells that have been separated via magnetic particles remain behind, and are available for additional applications until the point in time of switching off/removing the magnetic field.

TABLE 9
Antibody mixture 1

| Antigen | Clone | Concentration |
|---|---|---|
| Epith. Rel. Antigen | MOC-31 (Fa. Novocastra) | 1.25 μL/10⁶ cells |
| Epithelial antigen | Ber-EP 4 (Fa. DAKO) | 0.924 μg/10⁶ cells |

However, tumor cells were recognized with high specificity quite generally by means of the antibody mixture in Table 9. This is based on the selective expression of certain surface proteins that differentiate cancer cells from other cells.

In comparison to the separately used antibodies, an increased sensitivity during cell separation was demonstrated quite invariably, and independently of the method used, as a result of the use of the antibody mixture. This is shown in FIG. 3, whereby use was made in subdiagram A of magnetic particles that were coated with the antibody BER-EP4, and whereby use was made in subdiagram B of magnetic particles that were coated with the antibody MOC-31, and whereby use was made in subdiagram C of a mixture comprising particles that were each separately coated with an antibody.

A total of four measurements, in which, respectively, 1, 10, 100, or 1000 carcinoma cells in 10 mL of blood were inoculated, were carried out for each of the antibodies or antibody mixtures. Lanes 1a through 4a, 1b through 4b, and 1c through 4c then show the detection of RNA following RNA preparation and RT-PCR with tumor marker-specific primers, as described above, for samples with a volume of 1 μL in each case. FIG. 3 was obtained by means of electrophoretic separation in an Agilent™ Bioanalyzer 2100 in accordance with data from the manufacturer.

When using magnetic particles labeled with merely one antibody as in FIGS. 3A and 3B, positive detection was possible only with a quantity amounting to 1000 cells. When using an antibody mixture as in FIG. 3C, detection was achieved with only 100 cells, i.e. an increase in sensitivity by a factor of 10.

In this example, experimental results have been shown that do not represent the maximum possible sensitivity but, by way of example, they demonstrate the increase in sensitivity that is achievable with the procedure in accordance with the invention.

FIG. 4 shows the detection of breast cancer cells via the simultaneous recognition of tumor cell markers GA733.2, MUC1, Her-2 and claudin-7. In this case, breast tumor cells in a sample were inoculated, whereby different cell lines, namely cell line 1 and cell line 2, were introduced. Enrichment of the tumor cells by means of antibody-coupled magnetic particles took place prior to the determination of the markers, whereby use was made of the antibodies BerEp4, HMTV.2 and GP1.4. FIG. 4 then shows that, for both cell lines, sure recognition can be demonstrated down to two cells per 5 mL by means of such a combination of the tumor markers that were to be recognized. A nonspecific reaction did not take place in this regard. In the following segment, the PCR conditions are illustrated for the different polymerase chain reactions shown in FIG. 4 through FIG. 8 in order to detect tumor markers from breast cancer cells.

Standard, FIG. 4

| Marker | Primer | Concentration |
|---|---|---|
| Actin | sense | 0.1 μM |
| | antisense | 0.1 μM |
| Claudin-7 | sense | 0.3 μM |
| | anrtisense | 0.3 μM |
| Her-2 | sense | 0.3 μM |
| | antisense | 0.3 μM |
| MUC1 | sense | 0.4 μM |
| | antisense | 0.4 μM |
| GA733.2 | sense | 1 μM |
| | antisense | 1 μM |

Cycles: 35

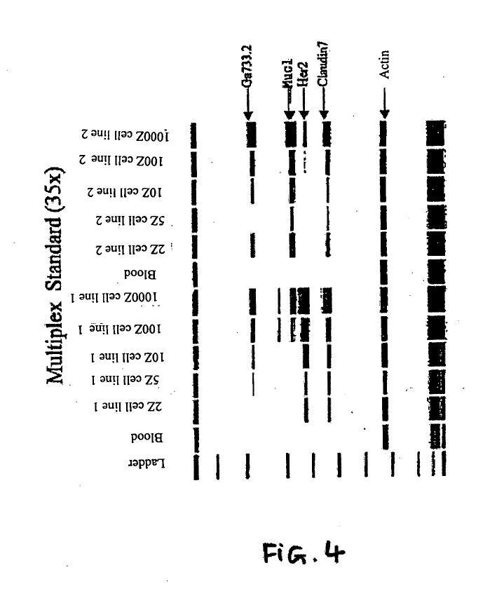

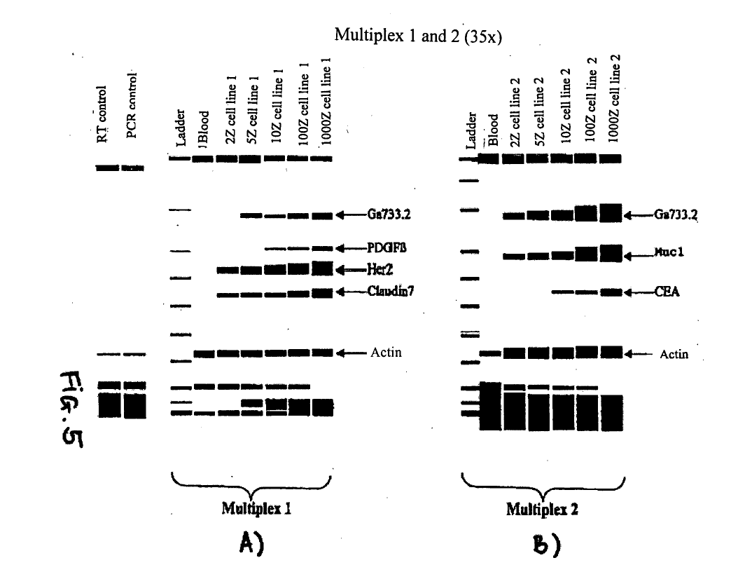

Multiplex 1, FIG. 5A

| Marker | Primer | Concentration |
|---|---|---|
| Actin | sense | 0,1 μM |
| | antisense | 0,1 μM |
| Claudin-7 | sense | 0,3 μM |
| | antisense | 0,3 μM |
| PDGF-β | sense | 1,2 μM |
| | antisense | 1,2 μM |
| Her-2 | sense | 0,3 μM |
| | antisense | 0,3 μM |
| GA733.2 | sense | 1 μM |
| | antisense | 1 μM |

Cycles: 35

Multiplex 2, FIG. 5B

| Marker | Primer | Concentration |
|---|---|---|
| Actin | sense | 0,1 μM |
| | antisense | 0,1 μM |
| GA733.2 | sense | 1 μM |
| | antisense | 1 μM |
| MUC1 | sense | 0,4 μM |
| | antisense | 0,4 μM |
| CEA | sense | 2 μM |
| | antisense | 2 μM |

Cycles: 35

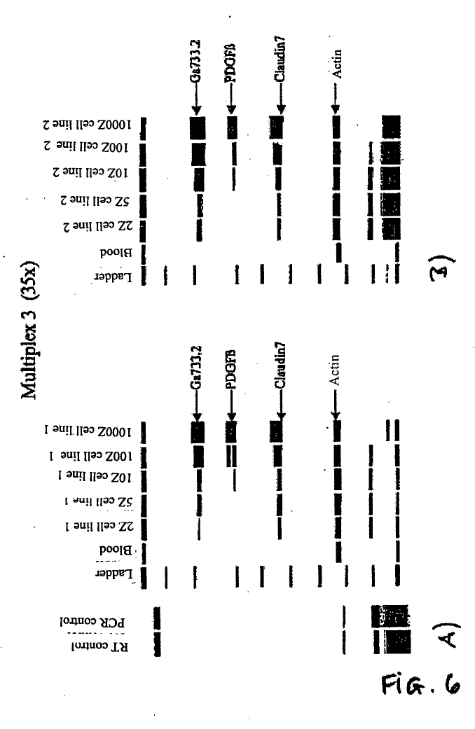

Multiplex 3, FIG. 6

| Marker | Primer | Concentration |
|---|---|---|
| Actin | sense | 0,1 μM |
| | antisense | 0,1 μM |
| GA733.2 | sense | 1 μM |
| | antisense | 1 μM |
| PDGF-β | sense | 1,2 μM |
| | antisense | 1,2 μM |
| Claudin-7 | sense | 0,3 μM |
| | antisense | 0,3 μM |

Cycles: 35

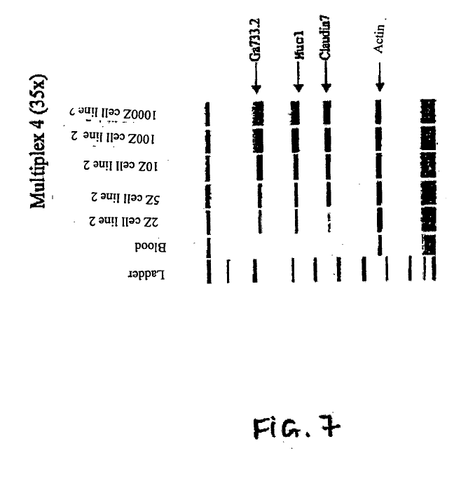

Multiplex 4, FIG. 7

| Marker | Primer | Concentration |
|---|---|---|
| Actin | Sense | 0,1 μM |
| | Antisense | 0,1 μM |
| Claudin-7 | Sense | 0,3 μM |
| | Antisense | 0,3 μM |
| MUC1 | Sense | 0,4 μM |
| | Antisense | 0,4 μM |
| GA733.2 | Sense | 1 μM |
| | Antisense | 1 μM |

Cycles: 35

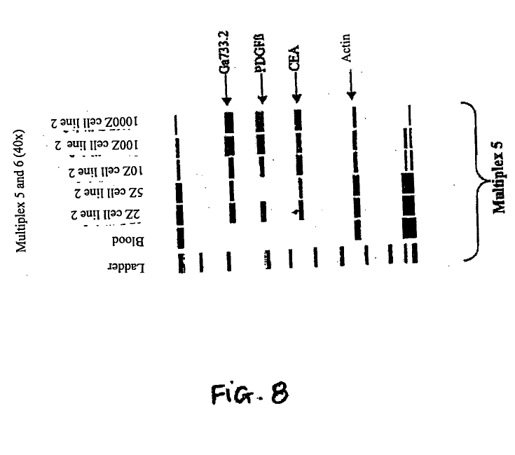

Multiplex 5, FIG. 8A

| Marker | Primer | Concentration |
|---|---|---|
| Actin | sense | 0,05 μM |
| | antisense | 0,05 μM |
| GA733.2 | sense | 0,05 μM |
| | antisense | 0,05 μM |
| PDGF-β | sense | 005 μM |
| | antisense | 0,05 μM |
| CEA | sense | 0,7 μM |
| | antisense | 0,7 μM |

Cycles: 40

FIG. 5, likewise, shows the detection of breast tumor cells, whereby the combinations comprising the markers GA733.2, PDGF-β, Her-2 and claudin-7 (FIG. 5A) or GA733.2, MUCL and CEA were recognized. Highly sensitive recognition takes place once again down to two cells per 5 mL sample, whereas no nonspecific detection reactions occurred in a control blood sample.

FIG. 6 illustrates the use of the marker combination GA733.2, PDGF-β and claudin-7 (FIG. 6A), or GA733.2, PDGF-β and claudin-7 (FIG. 6B) for a first cell line (FIG. 6A), or a second cell line (FIG. 6B). Highly specific detection again takes place without nonspecific reactions, whereby cell line 2 can be detected better than cell line 1 in this case. FIG. 7 again illustrates a detection procedure by means of the combination of the markers GA733.2, MUC1 and claudin-7. Highly specific detection takes place for cell line 2 down to two cells per 5 mL sample for each individual marker and, in particular, for the combination of the markers, without nonspecific detection reactions.

FIG. 8 shows the detection procedure by means of the marker combination GA733.2, PDGF-β and CEA for cell line 2. Highly specific detection again takes place down to two cells per 5 mL sample without nonspecific detection reactions.

The diagnosis kit in accordance with the invention and the procedure in accordance with the invention also make it possible to subsequently use the sorted and separated cells further as desired. For example, these can be inserted into a suitable cell culture medium where they can be cultivated in situ.

Since the cells are intact following separation, the properties of the cell membrane and of the cell nucleus are also conserved. This opens up the possibility of microscopically investigating the expression of additional surface markers, and of carrying out chromosome analyses as well. The sorted cells are applied to microscope slides for this purpose. The detection of additional surface markers can take place cytochemically or via fluorescence microscopy. Likewise, genetic analyses can be carried out such as, for example, chromosome analyses by means of FISH (fluorescence in situ hybridization), or via karyogram compilation.

1. Procedure for the diagnosis or monitoring of breast cancer in humans, characterized in that the presence or absence of at least three different mRNAs is recognized in a human blood sample, whereby these code for various members of the tumor marker proteins GA733.2, MUC1 and Her-2/neu and, if at least one of the mRNAs is present, conclusions are drawn in regard to the presence of mammary carcinoma cells in the sample of blood, and hence in regard to possible metastasis.

2. Procedure in accordance with claim 1, characterized in that the presence or absence of additional mRNAs is recognized that code for various members of the tumor marker proteins claudin-7, CK20, MAGE-3, stanniocalcin, EGF-R and/or CEA.

3. Procedure in accordance with the preceding claim, characterized in that the mRNA associated with the genes GA733.2, MUC1, Her-2/neu and claudin-7 is recognized.

4. Procedure in accordance with one of the preceding claims, characterized in that tumor cells from the blood sample are separated or concentrated, and the detection procedure is done using these tumor cells.

5. Procedure in accordance with the preceding claim, characterized in that the mammary carcinoma cells are separated or concentrated by means of antibodies, which are generally specific for tumor cells, and/or by means of antibodies specific for mammary carcinoma cells or by means of mixtures of such antibodies.

6. Procedure in accordance with the preceding claim, characterized in that the antibodies or antibody derivatives used for separating mammary tumor cells have binding sites that bind to the epitopes of an epithelial antigen of an epithelial membrane antigen and/or of the antigen MUC1.

7. Procedure in accordance with the preceding claim, characterized in that use is made of MOC-31 and/or Ber-EP4, or a mixture of these, as the antibody.

8. Device in accordance with one of the preceding claims, characterized in that use is made of 131-11741, E29, GP1.4 and/or HMPV.2, or a mixture of all of these, as the antibody.

9. Procedure in accordance with one of the two preceding claims, characterized in that the mammary carcinoma cells are separated or concentrated by means of antibodies bound to magnetic particles.

10. Procedure in accordance with claim 4, characterized in that the mammary carcinoma cells are separated or concentrated by means of fluorescence-activated continuous flow cytometry, density gradient centrifugation, and/or centrifugation following erythrocyte lysis.

11. Procedure in accordance with claim 10, characterized in that leukocytes in the blood sample are pelletized by centrifugation.

12. Procedure in accordance with claim 10, characterized in that the RNA-containing components of the sample are concentrated via lysis of the erythrocytes that are contained in it, together with subsequent pelletization of the nonlysed leukocytes.

13. Procedure in accordance with claim 10, characterized in that the RNA-containing components are concentrated by at least one density gradient centrifugation of the blood sample in order to separate and harvest the mononuclear blood cells that are contained in it.

14. Procedure in accordance with one of claims 10 through 13, characterized in that the harvested mononuclear blood cells are labeled with fluorescence-labeled antibodies and are separated and harvested by means of fluorescence-activated cell sorting (FACS) of the sample.

15. Procedure in accordance with one of claims 10 through 14, characterized in that the mononuclear cells from the harvested fraction are lysed, and the mRNA is separated.

16. Procedure in accordance with claim 1, characterized in that the RNA (total RNA or mRNA) is isolated directly and in a conventional manner from the whole blood sample.

17. Procedure in accordance with the preceding claim, characterized in that DNA digestion is carried out subsequently to isolation of the RNA.

18. Procedure in accordance with one of the preceding claims, characterized in that the harvested mRNA is reverse transcribed into cDNA, and the presence or absence of the cDNA that is assigned to the tumor marker protein is recognized.

19. Procedure in accordance with the preceding claim, characterized in that at least one predetermined segment of the cDNA is replicated by means of a polymerase chain reaction (“PCR”).

20. Procedure in accordance with the preceding claim, characterized in that one or more oligonucleotide pairs which exhibit the following sequences are used for replicating the cDNA:
AATCGTCAATGCCAGTGTACTTCA
and
TAACGCGTTGTGATCTCCTTCTGA
and/or
AGTCGGGCTCTGGAGGAAAAGAAA
and
GATCATAATTCCTCTGCACATAGG
and/or
AGAAATGACGCAAGAGCCTATGTA
and
AACTTGTGTGTGTTGCTGCGGTAT
and/or
TCAGCTTCTACTCTGGTGCACAAC
and
TGGTAGTAGTCGGTGCTGGGATCT
and/or
CCCAGTGTGTCAACTGCAGCCAGT
and
CAGATGGGCATGTAGGAGAGGTCA
and/or
GTCTTGCCGCCTTGGTAGCTTGCT
and
TGGACTTAGGGTAAGAGCGGGGTG
and/or
ATCTCCAAGGCCTGAATAAGGTCT
and
CCTCAGTTCCTTTTAATTCTTCAGT
and/or
CTCCAGCCTCCCCACTACCATGAA
and
TTGTCACCCAGCAGGCCATCGTAG
and/or
AACCCATGAGGCGGAGCAGAATGA
and
CGTTGGCGATGCATTTTAAGCTCT
and/or
TCTCTCTGCTGCTACCTGCGTCTG
and
GTTGGCGTTGGTGCGGTCTATGAG

21. Procedure in accordance with one of the preceding claims, characterized in that the mRNA of the protein β-actin is determined for internal control purposes.

22. Procedure in accordance with the preceding claim, characterized in that the mRNA for β-actin is reverse transcribed into cDNA, and a segment of the cDNA is replicated by means of a polymerase chain reaction.

23. Procedure in accordance with the preceding claim, characterized in that an oligonucleotide pair is used for replicating the cDNA of the β-actin, whereby the oligonucleotides of the pair exhibit the following sequences:
CTG GAG AAG AGC TAC GAG CTG CCT
and
ACA GGA CTC CAT GCC CAG GAA GGA.

24. Procedure in accordance with one of claims 19 through 23, characterized in that the replicated cDNA segment is digested via suitable restriction enzymes, and the presence or absence of the mRNA of a tumor marker protein is determined by means of the cDNA fragments that are produced.

25. Procedure in accordance with one of claims 19 through 23, characterized in that gel electrophoresis of the PCR products is carried out in order to detect the amplified cDNA segments.

26. Procedure in accordance with one of claims 19 through 23, characterized in that fragment analysis is carried out in order to detect the amplified cDNA segments.

27. Procedure in accordance with one of claims 19 through 23, characterized in that, during the course of the polymerase chain reaction, the fluorescence produced by the products is recognized and the formation of products is recognized (fluorescence-based real time PCR).

28. Procedure in accordance with one of claims 19 through 23, characterized in that use is made of a nucleotide microarray in accordance with one of claims 48 through 50 in order to detect the mRNA or cDNA.

29. Procedure in accordance with the preceding claim, characterized in that the PCR product is applied to a nucleotide microarray in accordance with one of claims 48 through 50 in order to detect the amplified cDNA.

30. Diagnosis kit for the diagnosis or monitoring of breast cancer via at least three pairs of oligonucleotides (reverse primers, forward primers), whereby the two oligonucleotides of each pair are suitable as primers for amplification by means of a polymerase chain reaction of, in each case, one of the two complementary strands of different DNA segments that are being sought, and whereby the DNA segments that are being sought are a part of the cDNA associated with various members of the genes GA733.2, MUC-1 and Her-2/neu.

31. Diagnosis kit in accordance with claim 30, characterized in that it contains at least one additional pair of oligonucleotides (reverse primers, forward primers), whereby the two oligonucleotides of each pair are suitable as primers for amplification by means of a polymerase chain reaction of, in each case, one of the two complementary strands of a DNA segment that is being sought, and whereby the DNA segment that is being sought is a part of the cDNA associated with one of the tumor marker proteins CK20, EGF-R, CEA and stanniocalcin, or CK20, MAGE-3, claudin-7, and/or PDGF-β.

32. Diagnosis kit in accordance with claim 30, characterized in that it contains at least four pairs of oligonucleotides (reverse primers, forward primers), whereby the two oligonucleotides of each pair are suitable as primers for amplification by means of a polymerase chain reaction of, in each case, one of the two complementary strands of different DNA segments that are being sought, and whereby the DNA segments that are being sought are, in each case, a part of the cDNA associated with the tumor marker proteins GA733.2, MUC-1, Her-2/neu and claudin-7.

33. Diagnosis kit in accordance with one of the two preceding claims, characterized in that it contains an additional pair of oligonucleotides that are, in each case, suitable as primers for the amplification of at least one segment of the two complementary strands of the cDNA associated with the protein β-actin for internal control purposes.

34. Diagnosis kit in accordance with one of claims 30 through 33, characterized in that the two oligonucleotides of a pair exhibit the following sequences in a pair-wise manner:
AATCGTCAATGCCAGTGTACTTCA
and
TAACGCGTTGTGATCTCCTTCTGA
and/or
AGTCGGGCTCTGGAGGAAAAGAAA
and
GATCATAATTCCTCTGCACATAGG
and/or
AGAAATGACGCAAGAGCCTATGTA
and
AACTTGTGTGTGTTGCTGCGGTAT
and/or
TCAGCTTCTACTCTGGTGCACAAC
and
TGGTAGTAGTCGGTGCTGGGATCT
and/or
CCCAGTGTGTCAACTGCAGCCAGT
and
CAGATGGGCATGTAGGAGAGGTCA
and/or
GTCTTGCCGCCTTGGTAGCTTGCT
and
TGGACTTAGGGTAAGAGCGGGGTG
and/or
ATCTCCAAGGCCTGAATAAGGTCT
and
CCTCAGTTCCTTTTAATTCTTCAGT
and/or
CTCCAGCCTCCCCACTACCATGAA
and
TTGTCACCCAGCAGGCCATCGTAG
and/or
AACCCATGAGGCGGAGCAGAATGA
and
CGTTGGCGATGCATTTTAAGCTCT
and/or
TCTCTCTGCTGCTACCTGCGTCTG
and
GTTGGCGTTGGTGCGGTCTATGAG.

35. Diagnosis kit in accordance with one of claims 30 through 34, characterized in that, in each case, at least one of the two oligonucleotides of a pair of oligonucleotides is labeled with fluorophores.

36. Diagnosis kit in accordance with the preceding claim, characterized in that the oligonucleotides of different pairs are labeled with different fluorophores.

37. Diagnosis kit in accordance with one of claims 30 through 36, characterized in that, in order to amplify the cDNA associated with β-actin, it contains a pair of oligonucleotides with the following sequences:
CTG GAG AAG AGC TAG GAG CTG GGT
and
ACA GGA CTC CAT GGG GAG GAA GGA.

38. Diagnosis kit in accordance with the preceding claim, characterized in that, in each case, at least one of the two oligonucleotides of the pair is labeled with fluorophores in order to amplify the cDNA associated with β-actin.

39. Diagnosis kit in accordance with one of claims 30 through 38, characterized in that it contains the substances that are required for carrying out a polymerase chain reaction.

40. Diagnosis kit in accordance with one of claims 30 through 39, characterized in that it contains the following as the substances that are required for carrying out a polymerase chain reaction: a buffer solution, magnesium chloride, deoxynucleotide triphosphates as well as a heat-stable polymerase.

41. Diagnosis kit in accordance with the preceding claim, characterized in that it contains a polymerase from Thermus aquaticus (Taq polymerase) as the heat-stable polymerase.

42. Diagnosis kit in accordance with one of claims 30 through 41, characterized in that, as a positive control, it contains a DNA sample with the DNA segment that is being sought in each case.

43. Diagnosis kit in accordance with one of claims 30 through 42, characterized in that it contains:
instructions for carrying out the polymerase chain reaction and/or
instructions for carrying out a fragment analysis.

44. Diagnosis kit in accordance with one of claims 30 through 43, characterized in that it contains a schematic arrangement for evaluating the measurement results.

45. Diagnosis kit in accordance with one of claims 30 through 44, characterized in that it contains a microarray (DNA chip), whereby the array has a number of cells (fields) which are separated from one another, and an oligonucleotide, which hybridizes with the DNA segment that is being sought, is arranged in at least one cell of the microarray.

46. Diagnosis kit in accordance with the preceding claim, characterized in that an additional oligonucleotide is arranged in at least one additional cell of the microarray, and the sequence of the nucleotide arranged in said cell differs from the sequence of the additional oligonucleotide.

47. Diagnosis kit in accordance with one of the two preceding claims, characterized in that an oligonucleotide is arranged in at least two cells in each case, whereby the oligonucleotides are arranged in different cells hybridize in each case with the different DNA segments that are being sought.

48. Microarray for the diagnosis or monitoring of breast cancer, e.g. a DNA chip, with an arrangement of several cells that are separated from one another, characterized in that, in each case, different oligonucleotides are arranged in at least three cells, whereby these oligonucleotides hybridize with a DNA segment that is part of the cDNA associated with the three different tumor marker proteins GA733.2, MUC-1 and Her-2/neu.

49. Microarray in accordance with claim 48, characterized in that different oligonucleotides are arranged in at least four cells in each case, whereby these oligonucleotides hybridize in each case with four different DNA segments that are part of the cDNA of the tumor marker proteins GA733.2, MUC-1, Her-2/neu and claudin-7.

50. Microarray in accordance with claim 48 or 49, characterized in that oligonucleotides are arranged in additional cells, whereby these oligonucleotides hybridize with DNA segments that are part of the cDNA of the tumor marker proteins CK20, EGF-R, CEA, stanniocalcin, MAGE-3 and/or PDGF-β.

51. Use of a diagnosis kit, a microarray and/or a procedure in accordance with one of the preceding claims for the diagnosis of diseases or metastasis or for monitoring in cases of breast cancer.


SEQUENCE LISTING (22 sequences)

SEQ ID NO: 1 - DNA, 24 bp, Artificial
  Oligonucleotide
aatcgtcaatgccagtgtacttca

SEQ ID NO: 2 - DNA, 24 bp, Artificial
  Oligonucleotide
taacgcgttgtgatctccttctga

SEQ ID NO: 3 - DNA, 24 bp, Artificial
  Oligonucleotide
agtcgggctctggaggaaaagaaa

SEQ ID NO: 4 - DNA, 24 bp, Artificial
  Oligonucleotide
gatcataattcctctgcacatagg

SEQ ID NO: 5 - DNA, 24 bp, Artificial
  Oligonucleotide
agaaatgacgcaagagcctatgta

SEQ ID NO: 6 - DNA, 24 bp, Artificial
  Oligonucleotide
aacttgtgtgtgttgctgcggtat

SEQ ID NO: 7 - DNA, 24 bp, Artificial
  Oligonucleotide
tcagcttctactctggtgcacaac

SEQ ID NO: 8 - DNA, 24 bp, Artificial
  Oligonucleotide
tggtagtagtcggtgctgggatct

SEQ ID NO: 9 - DNA, 24 bp, Artificial
  Oligonucleotide
cccagtgtgtcaactgcagccagt

SEQ ID NO: 10 - DNA, 24 bp, Artificial
  Oligonucleotide
cagatgggcatgtaggagaggtca

SEQ ID NO: 11 - DNA, 24 bp, Artificial
  Oligonucleotide
gtcttgccgccttggtagcttgct

SEQ ID NO: 12 - DNA, 24 bp, Artificial
  Oligonucleotide
tggacttagggtaagagcggggtg

SEQ ID NO: 13 - DNA, 24 bp, Artificial
  Oligonucleotide
atctccaaggcctgaataaggtct

SEQ ID NO: 14 - DNA, 25 bp, Artificial
  Oligonucleotide
cctcagttccttttaattcttcagt

SEQ ID NO: 15 - DNA, 24 bp, Artificial
  Oligonucleotide
ctccagcctccccactaccatgaa

SEQ ID NO: 16 - DNA, 24 bp, Artificial
  Oligonucleotide
ttgtcacccagcaggccatcgtag

SEQ ID NO: 17 - DNA, 24 bp, Artificial
  Oligonucleotide
aacccatgaggcggagcagaatga

SEQ ID NO: 18 - DNA, 24 bp, Artificial
  Oligonucleotide
cgttggcgatgcattttaagctct

SEQ ID NO: 19 - DNA, 24 bp, Artificial
  Oligonucleotide
tctctctgctgctacctgcgtctg

SEQ ID NO: 20 - DNA, 24 bp, Artificial
  Oligonucleotide
gttggcgttggtgcggtctatgag

SEQ ID NO: 21 - DNA, 24 bp, Artificial
  Oligonucleotide
ctggagaagagctacgagctgcct

SEQ ID NO: 22 - DNA, 24 bp, Artificial
  Oligonucleotide
acaggactccatgcccaggaagga